(19) United States
(12) Patent Application Publication
(10) Pub. No.: US 2007/0272236 A1
(43) Pub. Date: Nov. 29, 2007

(54) Blinding Device

(75) Inventor: Jutta Pfeiffer, Langenargen (DE)

Correspondence Address: NATH & ASSOCIATES PLLC, 112 South West Street, Alexandria, VA, 22314, US

(73) Assignee: ALTANA PHARMA AG, Konstanz (DE)

(21) Appl. No.: 11/587,945

(22) PCT Filed: May 2, 2005
(86) PCT No.: PCT/EP05/51992

§ 371 Date: Nov. 14, 2006

(30) Foreign Application Priority Data
May 3, 2004 (DE) 20 2004 007 115.0

(51) Int. Cl. A61M 11/00

(52) U.S. Cl. 128/200.21

(57) ABSTRACT

The present invention relates to a cap (1) which is placed onto an aerosol container (2) in order to permit blinding of clinical studies.

### TECHNICAL FIELD OF THE INVENTION

The invention relates to a device which permits blinding of a pressurized container for inhalation.

### PRIOR ART

Many active substances for treatment of diseases of the airways, such as asthma, are provided as preparations in pressurized containers from which they are administered into the lungs. Such a pharmaceutical product is made up, for example, of an aerosol container (pressurized container), which has a dispensing valve, and of a mouthpiece in which the container is received. The valve stem of the dispensing valve is received in a nozzle section of the mouthpiece.

The bases of the aerosol containers which hold test medication and which are used in clinical studies often have imprints which allude to the nature of the product or to the manufacturer and which could thus lead to unblinding of the study. When conducting clinical studies with such products, it is therefore necessary to make the bases unviewable, i.e. to ensure blinding of the product.

This has hitherto been achieved mainly by covering the bases with materials such as adhesive or talcum powder. However, this method proves very problematic because the blinding has to be done manually, thus taking up a great deal of time (manual mixing and setting of the adhesive). The correct mixing ratio with talcum powder is also difficult to obtain, and this in practice also results in products of non-uniform appearance and in high reject rates. Vapours given off by the adhesives also make the blinding process very unpleasant.

DD 255260 is related to the blinding of vials containing sterile pharmaceutical compositions such as solutions, suspensions, freeze-dried products or powdered products.

U.S. Pat. No. 6,544,250 is related to a device for blinding the administration of nonsolid pharmaceutical administration forms. The device is in particular an opaque container which has at least two attachments, one of which is intended for connection to a dispenser for the pharmaceutical administration form, and the other of which is intended for connection to an applicator, the inside of said container having means for holding back the pharmaceutical administration form.

### DISCLOSURE OF THE INVENTION

The object of the present invention is to make available a blinded product which comprises an aerosol container, can be produced in a simple manner and avoids the abovementioned disadvantages of previous products.

According to the invention, the object is achieved by an opaque cap which is placed with an exact fit onto the base of the aerosol container and whose inner edge automatically wedges with the aerosol container when fitted.

The subject of the invention is therefore a pharmaceutical product comprising an aerosol container, a cap being placed with a form fit onto the base of the aerosol container, and its inner edge automatically wedging with the aerosol container.

Further embodiments according to the invention are set out in the patent claims.

The aerosol container is preferably a pressurized container with a dispensing valve at the end directed away from the base.

The pharmaceutical product preferably comprises a mouthpiece in which the aerosol container is received.

According to the invention, the cap is preferably a cap made of metal, for example stainless steel. However, other materials such as plastic are also conceivable. Production is by methods known to the skilled person, for example by deep-drawing of sheet-metal strips using a suitable drawing and cutting tool.

According to the invention, the cap is designed as a hollow cylinder which is open at one end and has an opaque base at the other end. According to the invention, the height of the cap is preferably chosen such that, when the aerosol is actuated (pressed down), the space between mouthpiece and pressurized container is not changed. This is important to ensure that the required air stream for the inhalation process is not impaired.

According to the invention, the cap has an internal diameter narrowing slightly in the direction of the open end of the cap. The cap can then be placed onto the base of the aerosol container by means of a slight pressure. The internal diameter of the cap narrowing slightly in the direction of the open end of the cap permits automatic, preferably irreversible wedging of the cap on the aerosol container. It is in this way possible to ensure permanent adherence of the cap over the base of the aerosol container.

The dimensions of the cap depend on the size of the pressurized container to be covered. The cap according to the invention preferably has a height of 3 to 10 mm, preferably 4 to 6 mm, and an internal diameter in the range of 19 to 25 mm, preferably 21 to 23 mm.

Illustrative embodiments of the invention are explained in more detail with reference to the drawings, in which:

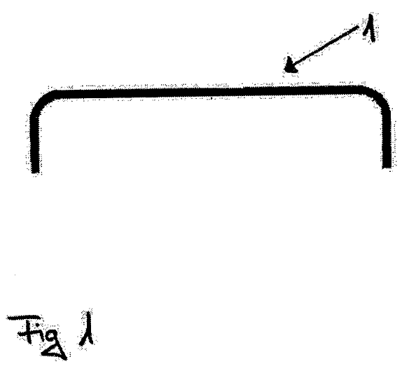

FIG. 1 shows a cap (1), according to the invention, made of stainless steel. The cap has a height of 5 mm and an internal diameter of 22.1 mm.

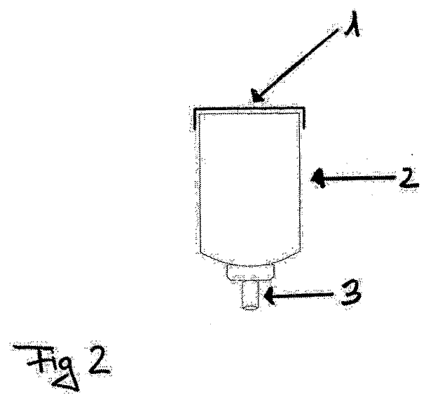

FIG. 2 shows an aerosol container (2) with dispensing valve (3) and fitted cap (1) (cross section).

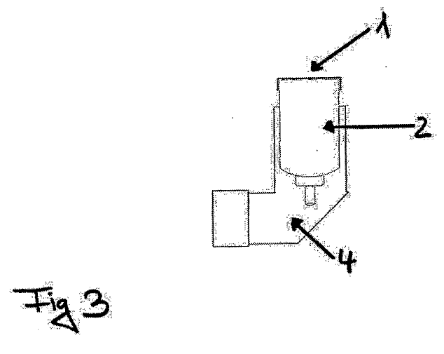

FIG. 3 shows an aerosol container (2) with fitted cap (1) in a mouthpiece (4) (cross section).

1. A pharmaceutical product comprising an aerosol container, a cap having an inner edge and an opaque base being placed with a form fit onto the base of the aerosol container, and the inner edge of the cap automatically wedging with the aerosol container.

2. The pharmaceutical product according to claim 1, in which the aerosol container is a pressurized container with a dispensing valve at the end directed away from the base.

3. The pharmaceutical product according to claim 1, in which the cap is made of metal.

4. The pharmaceutical product according to claim 1, in which the cap is designed as a hollow cylinder which is open at one end and has an opaque base at the other end.

5. The pharmaceutical product according to claim 4, in which the cap has an internal diameter narrowing slightly in the direction of the open end of the cap.

6. The pharmaceutical product according to claim 4, in which the cap has a height of 3 to 10 mm, and an internal diameter in the range of 19 to 25 mm.

7. The pharmaceutical product according to claim 6, in which the cap has a height of 5 mm and an internal diameter of 22.1 mm.

8. The pharmaceutical product according to claim 2, additionally having a mouthpiece in which the aerosol container is received.

9. The pharmaceutical product according to claim 4, in which the cap has a height of 4 to 6 mm, and an internal diameter in the range of 21 to 23 mm.
